(19) United States
(12) Patent Application Publication
(10) Pub. No.: US 2015/0374503 A1
(43) Pub. Date: Dec. 31, 2015

(54) IMPLANT FOR FUSION BETWEEN ADJACENT BONE BODIES

(71) Applicant: Bacterin International, Inc., Belgrade, MT (US)

(72) Inventors: Helena M. Lovick, Belgrade, MT (US); Craig S. Smith, Bozeman, MT (US); Dustin B. Byrd, Bozeman, MT (US); Todd R. Meyer, Bozeman, MT (US)

(73) Assignee: BACTERIN INTERNATIONAL, INC., Belgrade, MT (US)

(21) Appl. No.: 14/755,783

(22) Filed: Jun. 30, 2015

Related U.S. Application Data
(60) Provisional application No. 62/019,376, filed on Jun. 30, 2014.

(51) Int. Cl. A61F 2/42

(52) U.S. Cl. CPC A61F 2/4241; A61F 2002/30062

(57) ABSTRACT

The invention relates to three-dimensional implants to facilitate fusion between two adjacent bone bodies and methods of manufacture thereof.

### CROSS REFERENCE TO RELATED APPLICATIONS

This application claims priority and the benefit under 35 U.S.C. §119(e) to U.S. Provisional Patent Application Ser. No. 62/019,376 filed Jun. 30, 2014, which is incorporated herein in its entirety by reference.

### FIELD OF THE INVENTION

The invention generally relates to a three-dimensional implant for fusion between two adjacent bone bodies. In some embodiments, the bone bodies are phalanges in hands and/or feet.

### DESCRIPTION OF THE PRIOR ART

Other implants for fusion between two bone bodies are known in the prior art. The majority of these apply to spinal and ankle fusions. There remains a need for novel implants to allow fusion between adjacent bone bodies in the phalanges of the feet and hands of a patient.

U.S. Pat. No. 6,443,987 entitled “Spinal Vertebral Implant,” which is incorporated by reference in its entirety, discloses a spinal fusion implant to be mounted between adjacent vertebrae to achieve fusion there between. The spinal implant disclosed contains serrated sides for gripping the adjacent vertebrae upon placement. U.S. Pat. No. 7,922,765 entitled “Systems and Methods for the Fixation or Fusion of Bone,” which is incorporated by reference in its entirety, discloses methods for use of various bone fixation/fusion devices to be placed between bones to be fused. U.S. Publication No. 2010/0036439 entitled “Small Joint Fusion Implant,” discloses a two-component system for joining two bones, in which a first component is threaded with an annular mating end. The second component is threaded with the opposite handedness to the threads of the second component. U.S. Publication No. 2013/0150965 entitled “Fusion Implant,” which is incorporated by reference in its entirety, discloses an implant that facilitates the fusion between two bone parts. Various design elements of the implant are disclosed.

The invention discloses products that are advantageous over this art as discussed below.

### SUMMARY OF THE INVENTION

The disclosed invention is directed to a one-piece, three-dimensional implant for fusion between two adjacent bones, for example the phalanges of the feet and/or hands, of a patient. The implants are shaped to allow the curvature and structure of the phalanges to be maintained after bone fusion. The implants are composed of biocompatible materials. The implants can be composed of bone with an osteoconductive surface to facilitate bony fusion at the implantation site. The physical structure of the implants is designed to minimize insertion force while enhancing post-implantation pull-out resistance.

An aspect of the invention is a three-dimensional implant for fusing two adjacent bone bodies. The implant includes a body, at least one raised ridge along a surface of the body, and a raised stop. The body, the raised ridge, and/or the raised stop is made of a biocompatible material.

An aspect of the invention is a method of using the three-dimensional implant. The method includes preparing at least one cavity in a first bone and the second bone to be fused. The first bone and the second bone are adjacent. An implant, which includes a body, at least one raised ridge along the body of the implant, a raised stop along a length of the implant, a first end of the implant and a second end of the implant. At least one of the components of the implant is made from a biocompatible material. The raised stop has a first side and a second side. The first end of the implant in placed in the cavity of the first bone until the first side of the raised stop is in contact with a surface of the first bone. The second end of the implant is placed into a cavity of the second bone until the second side of the raised stop is in contact with a surface of the second bone.

### BRIEF DESCRIPTION OF THE FIGURES

FIG. 1 illustrates a perspective graphical view of a three-dimensional implant; and

FIG. 2 illustrates a graphical side view of a three-dimensional implant.

### DETAILED DESCRIPTION OF THE INVENTION

The invention relates to a three-dimensional implants and methods of using the implants.

“Allogeneic” or “allograft”, as used herein, refers to tissue derived from a non-identical donor of the same species.

“Antimicrobial agent” as used herein, broadly includes, but is not limited to, antibiotics, antimicrobials, antiseptics, and antifungals.

“Autogeneic” or “autograft”, as used herein, refers to tissue derived from and implanted into the same identical patient.

“End User”, as used herein, refers to a health care practitioner who performs the implantation of the bone implant into a patient.

“Patient”, as used herein, refers to a living recipient of the bone implant of the invention.

“Xenogeneic” or “xenograft”, as used herein, is defined as tissue derived from a non-identical donor a different species.

An aspect of the invention is a three-dimensional implant for fusing two adjacent bone bodies. The implant includes at least one biocompatible material and at least one raised ridge along at least one surface of the body of the implant. The implant also includes a defined raised stop along a length of the implant.

The raised stop can be at any location along the implant. In some embodiments, the raised stop can be approximately at the midline of the length of the implant. In some embodiments, the raised stop can be at a length approximately one third from an end of the implant. In some embodiments, the raised stop can be at an angle relative to an end of the implant. The angle can be between about 0° to about 15°, including any sub-range or particular value within these endpoints. The raised stop can be substantially circular in shape, but in some embodiments, can be oval, square, octagonal or other shapes.

The raised ridges can be wave-like ridges. Multiple ridges can be used, such that the ridges continue evenly spaced along the length of the implant. In some embodiments, the ridges can be intermittently located along the length of the implant. In some embodiments, at least one raised ridge can be irregularly spaced along the implant. In some embodiments, the ridge can be continuous along the length of the implant (for example in a sinusoidal repeating pattern). The ridges can be used to limit rotation of the implant during use. The ridges can be substantially circular in shape, but in some embodiments, can be wave-like, oval, square, octagonal or other shapes. In some embodiments, the shape of the ridges in combination with the valleys on the body of the implant can have an elevation gain of between about 3-30%, including any sub-range or particular value within these end points. In some embodiments, the elevation gain can be between about 5-25%, or about 10-20%. The radius of curvature of the valleys to the ridges can be between about 0.1 mm to 1 mm, including any sub-range or particular value within these end points. In some embodiments, the radius of curvature of the valleys to the ridges can be between about 0.2 mm to 0.8 mm, about 0.3 mm to 0.6 mm. The radius of curvature of the rise of the ridges can be between about 0.1 mm to 1 mm, including any sub-range or particular value within these end points. In some embodiments, the radius of curvature or the rise of the ridges can be between about 0.2 mm to 0.8 mm, or about 0.3 mm to 0.6 mm. The top of the ridges can have substantially flat sections. The length of the substantially flat sections can be between about 0.01 mm to 0.05 mm, including any sub-range or particular value within these end points. In some embodiments, the length of the substantially flat sections can be between about 0.015 mm to 0.045 mm, or about 0.02 mm to 0.04 mm.

The implant can be composed of two sides on either end of the raised stop. The implant sides can be the same material, or can be different materials. The implant sides can be oriented straight with respect to each other (180°) or bent at an angle from between about 140° to about 180°, including any sub-range or value within these end points. In some embodiments, the angle between the implant sides can be between about 160° to about 178°, or between about 165° to about 175°.

The length of the implants can vary from between about 3 mm to about 80 mm, or any sub-range or particular value within these end points. In some embodiments, the length of the implant can be between about 5 mm to about 60 mm, or between about 10 mm to about 140 mm. The cross-sectional height (e.g., diameter) of the implants can range from between about 0.2 mm to about 10 mm, including any sub-range or particular value within these end points. In some embodiments, the cross-sectional height can be between about 0.5 mm to about 8 mm, or between about 1 mm to about 6 mm. The valleys' cross-sectional areas (e.g., the minimum diameter of the body) on each side of the raised stop of the implant can be the same or can be different. Furthermore, the ridges' cross-sectional areas (e.g., the maximum diameter of the body) on each side of the raised stop of the implant can be the same or can be different. The cross-sectional area of the raised stop can be between about 20% to about 40% larger, including any sub-range or particular value within these end points, than the cross-sectional area of at least one cross-sectional area of the implant.

The implants can be composed of various biocompatible materials including, but not limited to, metals, polymers, plastics, biological tissues, and combinations thereof. In some embodiments, the implants can be composed of bone. The bone can be selected from cortical bone, cancellous bone, or combinations thereof. The bone can be allogeneic, autogeneic or xenogeneic in nature. The implants can be composed of mineralized, partially demineralized, fully demineralized bone, or a combination thereof. Demineralized bone matrix for use by the disclosed method can be prepared using any method or techniques known in the art (for examples of typical demineralization protocols, see U.S. Pat. Nos. 5,314,476, 6,855,169, and 8,574,825, each of which are incorporated in their entirety by reference). Partial demineralization can be performed by soaking the implants for an amount of time intended to only partially demineralize the surface of the implant. The acid for partial demineralization can be selected from the group of acids including, but not limited to, hydrochloric acid, hydrobromic acid, acetic acid and formic acid. Preferably, the acid will be hydrochloric acid. Demineralization times for partial demineralization of the implants can range from about 0.1 minute to about 60 minutes, from about 1 minute to about 30 minutes, from about 5 minutes to about 20 minutes. In some embodiments, the demineralization protocol for the bone implants can be optimized based on the bone type to provide the maximum level of growth factors, such as bone morphogenetic proteins (BMPs).

In some embodiments, the implant can include an additive material. Suitable additive materials include, but are not limited to, antimicrobial agents. Antimicrobial agents include, but are not limited to, bisbiguanides, silver nanoparticles, silver nitrate, silver oxide, silver salts, silver sulfadiazine, silver zeolites, triclosan, antifolates, aminoglycosides, carbapenems, cephalosporins, fluoroquinolines, glycopeptides, macrolides, monobactams, oxazolidones, penicillins, rifamycins, sulfonamides and tetracyclines. Other additives include, but are not limited to, cells, bone marrow aspirate, growth factors, and combinations thereof, which can be used with the antimicrobial agents, and/or the antibacterial agents.

In some embodiments, the implant includes voids throughout the implant. The void to implant material ratios can vary from between about 1:99 to about 1:1, including any sub-range or particular value within these end points. In some embodiments, the void to implant material ratio can be between about 1:19 to about 1:3.

The implants can be prepared by milling the bone or molding bone to the desired implant dimensions. Following shaping of the implants to the desired dimension, the bone implant can be dried using various method or techniques known in the art. The bone implants can be supplied dehydrated or hydrated to the end user. If the implants are supplied dehydrated, they can be fully or partially rehydrated by the end user prior to implantation. Suitable rehydrating liquids include, but are not limited to, aqueous buffers, balanced salt solutions, bone marrow aspirate, or combinations thereof. Suitable balanced salt solutions and aqueous buffers include, Hank's balanced salt solution, phosphate buffered saline, saline or combinations thereof

Preferably, the drying of the grafts can be performed by lyophilization wherein the dehydrated grafts' length can be reduced by less than about 10%, by less than about 5%, by less than about 3% of the grafts' original, non-dehydrated length.

The implant can be dehydrated. In some embodiments, the implant can have a residual moisture content of less than about 6%, including any sub-range between about 0% and about 6%, or any particular value within this range. In some embodiments, the implant can be fully hydrated such that the residual moisture can be greater than about 80%, including any sub-range between about 80% and about 100%, or any particular value within this range.

The implants can be supplied sterile (10⁻⁶ sterility assurance level) to the end user. Sterilization of the implants can be performed on hydrated or dehydrated implants. Sterilization can be achieved by exposure to gamma irradiation, e-beam irradiation, ethylene oxide, or peracetic acid. Following sterilization, the processed implants can exhibit break strengths of at least about 60%, at least about 70%, at least about 80% of pre-irradiation break strengths.

The three-dimensional implants can be implanted into a patient's phalanges to assist in fusion of two adjacent bone bodies. Suitable patient groups for implantation with the bone implants include animals with phalanges such as, but not limited to, primates, canines, and felines. In some embodiments, the patient can be a human.

An aspect of the invention is a method of using the three-dimensional implant. The implant includes at least one biocompatible material, and at least one raised ridge along at least one surface of the implant. The implant also includes a raised stop along a length of the implant.

The raised stop has a first side and a second side. The implant has at least two ends. The method includes preparing cavities in two adjacent bones to be fused. The implant is provided to a cavity on one of the two adjacent bones to be fused until the raised stop of the implant is in contact with a surface of the adjacent bone. The implant is then placed into the cavity of the second adjacent bone until the second side of the raised stop is in contact with a surface of the second adjacent bone.

The diameter of the cavity in either one or both of the bones can be smaller than the maximum cross-sectional area of the body of the implant to ensure a snug fit. The cross-sectional area of the cavity can be is between about 0.3 mm to about 0.01 mm, or any sub-range or particular value within these endpoints, smaller than the maximum cross-sectional of the implant. The diameter of the cavity in either one or both of the bones can be smaller than the minimum cross-sectional area of the body of the implant to ensure a snug fit. The cross-sectional area of the cavity can be is between about 0.3 mm to about 0.01 mm, or any sub-range or particular value within these endpoints, smaller than the minimum cross-sectional of the implant.

The raised ridges can be wave-like ridges. Multiple ridges can be used, such that the ridges continue evenly spaced along the length of the implant. In some embodiments, the ridges can be intermittently located along the length of the implant. In some embodiments, at least one raised ridge can be irregularly spaced along the implant. In some embodiments, the ridge can be continuous along the length of the implant (for example in a sinusoidal repeating pattern). The ridges can be used to limit rotation of the implant during use. The ridges can be substantially circular in shape, but in some embodiments, can be wave-like, oval, square, octagonal or other shapes. In some embodiments, the shape of the ridges in combination with the valleys on the body of the implant can have an elevation gain of between about 3-30%, including any sub-range or particular value within these end points. In some embodiments, the elevation gain can be between about 5-25%, or about 10-20%. The radius of curvature of the valleys to the ridges can be between about 0.1 mm to 1 mm, including any sub-range or particular value within these end points. In some embodiments, the radius of curvature of the valleys to the ridges can be between about 0.2 mm to 0.8 mm, about 0.3 mm to 0.6 mm. The radius of curvature of the rise of the ridges can be between about 0.1 mm to 1 mm, including any sub-range or particular value within these end points. In some embodiments, the radius of curvature or the rise of the ridges can be between about 0.2 mm to 0.8 mm, or about 0.3 mm to 0.6 mm. The top of the ridges can have substantially flat sections. The length of the substantially flat sections can be between about 0.01 mm to 0.05 mm, including any sub-range or particular value within these end points. In some embodiments, the length of the substantially flat sections can be between about 0.015 mm to 0.045 mm, or about 0.02 mm to 0.04 mm.

The implant can be composed of two sides on either end of the raised stop. The implant sides can be the same material, or can be different materials. The implant sides can be oriented straight with respect to each other (180°) or bent at an angle from between about 140° to about 180°, including any sub-range or value within these end points. In some embodiments, the angle between the implant sides can be between about 160° to about 178°, or between about 165° to about 175°.

The length of the implants can vary from between about 3 mm to about 80 mm, or any sub-range or particular value within these end points. In some embodiments, the length of the implant can be between about 5 mm to about 60 mm, or between about 10 mm to about 140 mm. The cross-sectional heights of the implants can range from between about 0.2 mm to about 10 mm, including any sub-range or particular value within these end points. In some embodiments, the cross-sectional heights can be between about 0.5 mm to about 8 mm, or between about 1 mm to about 6 mm. The minimum cross-sectional heights on each side of the implant can be the same or can be different. Furthermore, the maximum cross-sectional heights on each side of the implant can be the same or can be different.

The cross-sectional height of the raised stop can be between about 20% to about 40% larger, including any sub-range or particular value within these end points, than the cross-sectional height of at least one cross-sectional height of the implant. The raised stop includes a first side and a second side. The implant can come into contact with one of the adjacent bones on the first side of the raised stop, while the second side of the implant can come into contact with the other adjacent bone.

The implants can be composed of various biocompatible materials including, but not limited to, metals, polymers, plastics, biological tissues, and combinations thereof. In some embodiments the implants can be composed of bone. The bone can be selected from cortical bone, cancellous bone, or combinations thereof. The bone can be allogeneic, autogeneic or xenogeneic in nature. The implants can be composed of mineralized, partially demineralized, fully demineralized bone, or a combination thereof. Demineralized bone matrix for use by the disclosed method can be prepared using any method or techniques known in the art (for examples of typical demineralization protocols, see U.S. Pat. Nos. 5,314,476, 6,855,169, and 8,574,825, each of which are incorporated in their entirety by reference). Partial demineralization can be performed by soaking the implants for an amount of time intended to only partially demineralize the surface of the implant. The acid for partial demineralization can be selected from the group of acids including, but not limited to, hydrochloric acid, hydrobromic acid, acetic acid and formic acid. Preferably, the acid will be hydrochloric acid. Demineralization times for partial demineralization of the implants can range from about 0.1 minute to about 60 minutes, from about 1 minute to about 30 minutes, from about 5 minutes to about 20 minutes. In some embodiments, the demineralization protocol for the bone implants can be optimized based on the bone type to provide the maximum level of growth factors, such as bone morphogenetic proteins (BMPs).

In some embodiments, the implant can include an additive material. Suitable additive materials include, but are not limited to, antimicrobial agents. Antimicrobial agents include, but are not limited to, bisbiguanides, silver nanoparticles, silver nitrate, silver oxide, silver salts, silver sulfadiazine, silver zeolites, triclosan, antifolates, aminoglycosides, carbapenems, cephalosporins, fluoroquinolines, glycopeptides, macrolides, monobactams, oxazolidones, penicillins, rifamycins, sulfonamides and tetracyclines. Other additives include, but are not limited to, cells, bone marrow aspirate, growth factors, and combinations thereof, which can be used with the antimicrobial agents, and/or the antibacterial agents.

In some embodiments, the implant includes voids throughout the implant. The void to implant material ratios can vary from between about 1:99 to about 1:1, including any sub-range or particular value within these end points. In some embodiments, the void to implant material ratio can be between about 1:19 to about 1:3.

The implants can be prepared by milling the bone or molding bone to the desired implant dimensions. Following shaping of the implants to the desired dimension, the bone implant can be dried using various method or techniques known in the art. The bone implants can be supplied dehydrated or hydrated to the end user. If the implants are supplied dehydrated, they can be fully or partially rehydrated by the end user prior to implantation. Suitable rehydrating liquids include, but are not limited to, aqueous buffers, balanced salt solutions, bone marrow aspirate, or combinations thereof. Suitable balanced salt solutions and aqueous buffers include, Hank's balanced salt solution, phosphate buffered saline, saline or combinations thereof.

Preferably, the drying of the grafts can be performed by lyophilization wherein the dehydrated grafts' length can be reduced by less than about 10%, by less than about 5%, by less than about 3% of the grafts' original, non-dehydrated length.

The implant can be dehydrated. In some embodiments, the implant can have a residual moisture content of less than about 6%, including any sub-range between about 0% and about 6%, or any particular value within this range. In some embodiments, the implant can be fully hydrated such that the residual moisture can be greater than about 80%, including any sub-range between about 80% and about 100%, or any particular value within this range.

The implants can be supplied sterile (10⁻⁶ sterility assurance level) to the end user. Sterilization of the implants can be performed on hydrated or dehydrated implants. Sterilization can be achieved by exposure to gamma irradiation, e-beam irradiation, ethylene oxide, or peracetic acid. Following sterilization, the processed implants can exhibit break strengths of at least about 60%, at least about 70%, at least about 80% of pre-irradiation break strengths.

The three-dimensional implants can be implanted into a patient's phalanges to assist in fusion of two adjacent bone bodies. Suitable patient groups for implantation with the bone implants include animals with phalanges such as, but not limited to, primates, canines, and felines. In some embodiments, the patient can be a human.

An aspect of the invention is a method for the using the implants prepared in accordance with the embodiments of the invention. The implant fusion site in a patient is exposed, by way of example via an incision. Using a hollowing tool, such as a bone punch, burr, a drill bit, a reamer, or combinations thereof, each bone to be fused is hollowed at the implantation site. The depth and diameter of the prepared implantation sites are established based on the implant dimensions. The implant is then pressed fully into each prepared bone implantation site to the depth of the stop (if present) or to the depth of the side of the implant. The implantation site is then closed to allow the healing, bone fusion process to begin.

By way of non-limiting example, an approximately 2.75 mm diameter implant with sides of two lengths, about 5 mm and about 10 mm respectively, can be prepared. For the implantation site, voids of approximately the same dimensions as the implant, about 2.75 mm at depths of about 5 mm and about 10 mm respectively, would be prepared. If desired to provide a tighter implant fit, the implantation site void diameters can be slightly reduced from the implant dimensions (e.g., about 2.70 mm void diameter for an about 2.75 mm diameter implant, with similar dimension adjustments available for the length dimensions).

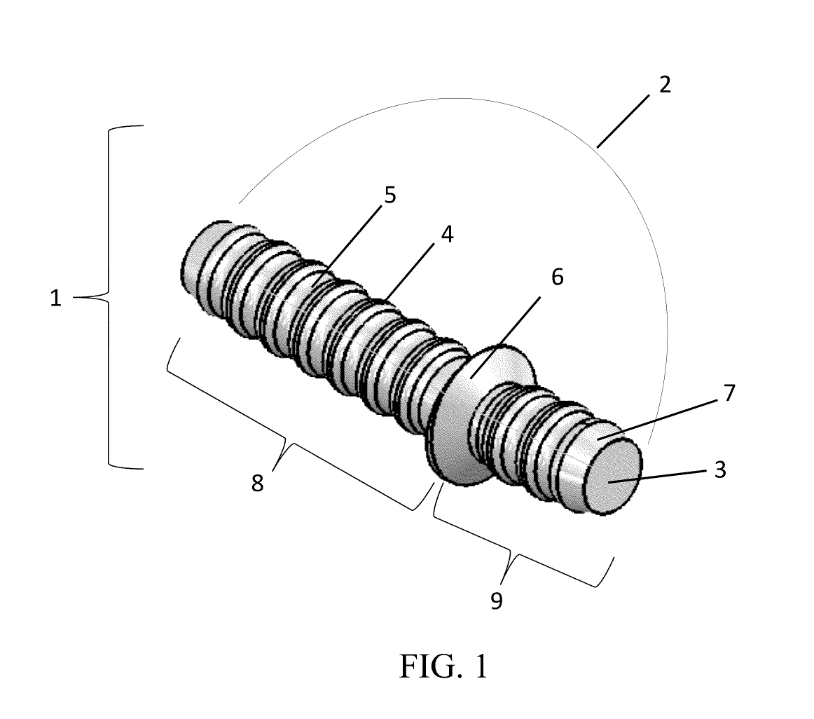

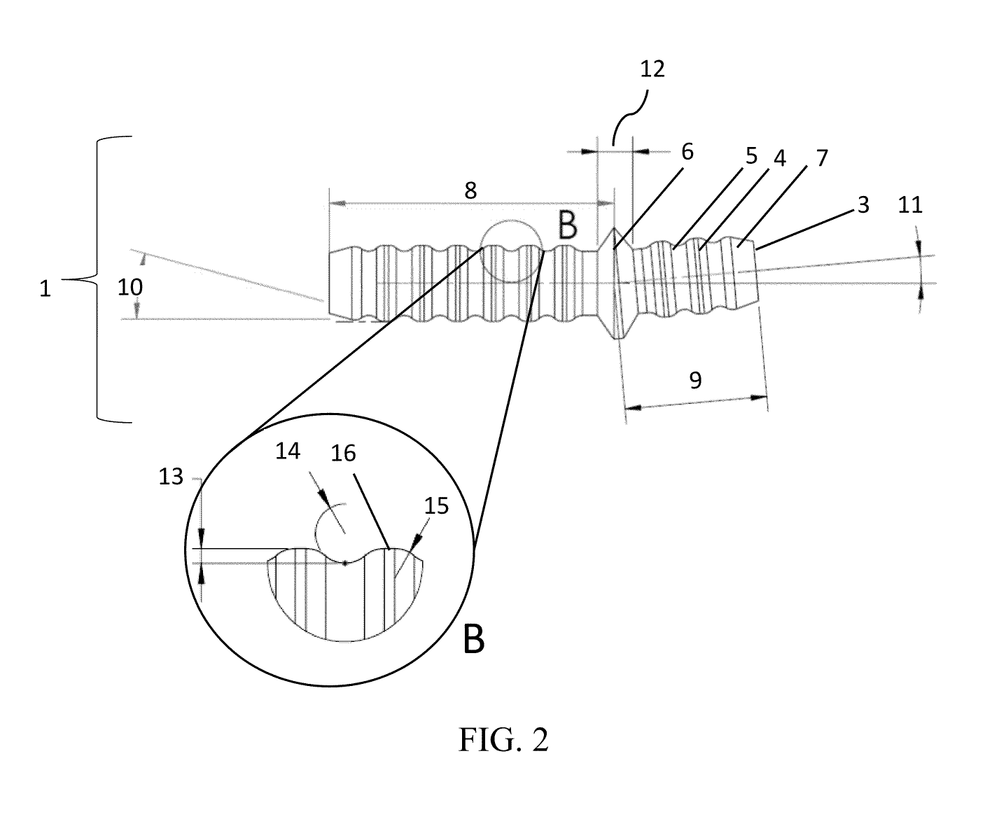

FIG. 1 illustrates a three-dimensional implant 1 in accordance with embodiments of the invention. The implant sides 8 and 9 can be oriented straight with respect to each other (180°) or bent at an angle 2 from between about 140° to about 179°, between about 160° to about 178°, or between about 165° to about 175°. For illustrative purposes, the implant 1 of FIG. 1 and FIG. 2 illustrates the sides 8 and 9 oriented at about 175°. The sides of the implant 8 and 9 can be equal in length or unequal in length. The length of side 9 can be about 10-90%, about 20-70%, about 30-60% of the length of side 8. The sides of the implant 8 and 9 can be tapered 10 towards the ends 3 of the implant 1 to facilitate placement during implantation. In some embodiments, only one side of the implant 1 can be tapered. In other embodiments, both sides of the implant 1 can be tapered. The angle of the tapered end(s) can vary from between about 1° to about 45°, between about 10° to about 30°, or between about 15° to about 25°. In some embodiments, the outer cross-sectional height of 8 and 9 can the same. In other embodiments, the outer cross-sectional height of 8 and 9 can be different. The length of the taper 10 can be about 0.2 mm to 10 mm, about 0.5 mm to 8 mm, about 1 mm to 5 mm. In some embodiments, the length of the each end's taper 10 can be equivalent to the entire length of the respective sides, side 8 or side 9. In some embodiments, the length of each side of the implant taper 10 can be equivalent. In some embodiments, the length of each side of the implant taper 10 can be different.

The implant 1 includes ridges 4 on the surface of the implant 1. The implant of FIG. 1 illustrates the ridges as sinusoidal-like ridges. The unique structure of the sinusoidal-like ridges 4 is designed to facilitate ease of implant insertion while subsequently limiting implant slippage, movement, or pistoning following implantation (i.e., enhanced pull-out resistance). Additionally the ridges 4 can provide additional surface area for osteoconductivity and implant incorporation into the patient. In between the ridges 4 on the implant 1, troughs or valleys 5 on the surface of the implant 1 can be present. The valleys 5 increase the osteoconductive surface of the implant and provide a surface for inclusion of other additives or materials to be added to the implant 1 prior to implantation. Suitable additives or materials to be added to the implant 1 prior to implantation include, but are not limited to, cells, bone marrow aspirate, growth factors, and combinations thereof. The sinusoidal-like shape of the ridges 4 in combination with the valleys 5 has an elevation gain 13 of about 3-30%, of about 5-25%, of about 10-20%. The radius of curvature 14 of the valleys 5 to the ridges 4 is about 0.1 mm to 1 mm, about 0.2 mm to 0.8 mm, about 0.3 mm to 0.6 mm. The radius of curvature 15 of the rise of the ridges 4 are about 0.1 mm to 1 mm, about 0.2 mm to 0.8 mm, about 0.3 mm to 0.6 mm. The top of the ridges 14 have substantially flat sections 16. The length of the substantially flat sections 16 is about 0.01 mm to 0.05 mm, about 0.015 mm to 0.045 mm, about 0.02 mm to 0.04 mm.

The ends of the implant 3 can be flat, concave or convex as desired. As illustrated in FIG. 1, the ends 3 can be approximately round. Other shapes of the ends 3 can include, but are not limited to, circles, ovals, regular polygons such as triangles, squares, pentagons, hexagons, heptagons, octagons, nonagons, and or decagons. The ends 3 of the implants can rounded or angled in the shape of polygons. In some embodiments, the ends 3 are angled to limit or prevent implant rotation following implantation. The shape of the ends 3 can include additional facets and sidedness in the form of angled edges 7. The angled edges 7 can include triangular or rectangular corners.

FIG. 2 illustrates a side-view of a three-dimensional implant 1 in accordance with embodiments of the invention. In some embodiments, a raised section or stop 6 can be located between the two sides 8 and 9 of the implant. The raised section can be anywhere along the length of the implant. In some embodiments, the raised section can be approximately at the midline of the length of the implant. The stop 6 can reduce or eliminate implant movement following implantation. Furthermore, the stop 6 can facilitate proper depth placement of the implant into an implantation site. The stop 6 can be symmetrical as illustrated in FIG. 2, or angled or hooked. The diameter of the stop 6 can be about 20%, about 30% or about 40% larger than the greater diameter of the two sides 8 and 9 of the implant. The cross-sectional shape of the stop 6 can be approximately square, circular, oval, or regular polygons such as triangles, pentagons, hexagons, heptagons, octagons, nonagons, and or decagons. The cross-sectional area 17 of the implant on one side of the raised stop may be less than or greater than the cross-sectional area 18 of the implant on the other side of the raised stop.

### EXAMPLE 1

### Insertion and Pull-Out Force of Implants

Using a 2.0 mm drill bit, voids 20 mm deep were prepared in a polyurethane foam block of 10 lb/ft³ density (Sawbones®, Vashon Island, Wash., USA). VisiJet® acrylic implants of a 2.5 mm outer diameter were inserted into the foam block. After insertion into the foam block, the pull-out testing of the implants was performed on an Instron 3343 Universal Testing Machine. The implants designed according to this invention with an internal diameter of 2.0 mm and an outer diameter of 2.5 mm had an average pull-out resistance of 33.1 Newtons.

The pull-out resistance of the invented implants is greater than the stated single-sided pull-out force for 3.5 mm diameter stainless steel ARROW-LOK™ implants (reported at 29.9 Newtons by Roman, S. R. “Comparing Kirschner wire fixation to a new device used for proximal interphalangeal fusion,” Podiatry Institute Update 2011: The Proceedings of the Annual Meeting of the Podiatry Institute).

The foregoing description of the invention has been presented for illustration and description purposes. However, the description is not intended to limit the invention to only the forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Consequently, variations and modifications commensurate with the above teachings and skill and knowledge of the relevant art are within the scope of the invention. The embodiments described herein above are further intended to explain best modes of practicing the invention and to enable others skilled in the art to utilize the invention in such a manner, or include other embodiments with various modifications as required by the particular application(s) or use(s) of the invention. Thus, it is intended that the claims be construed to include alternative embodiments to the extent permitted by the prior art.

1. A three-dimensional implant for fusing two adjacent bone bodies, comprising:
a body;
at least one raised ridge along at least one surface of the body; and
a raised stop along a length of the body, wherein the body, the at least one raised ridge, and the raised stop comprises at least a biocompatible material.

2. The three-dimensional of claim 1, wherein the raised stop is at an approximate midline of the body of the implant.

3. The three-dimensional of claim 1, wherein the raised stop is at approximately one third of a length of the body of the implant.

4. The three-dimensional of claim 1, wherein the at least one raised ridge is irregularly spaced on the body of the implant.

5. The three-dimensional of claim 1, wherein the at least one raised ridge is regularly spaced on the body of the implant.

6. The three-dimensional implant of claim 1, wherein the implant is angled at the raised stop.

7. The three-dimensional implant of claim 6, wherein an angle of the implant at the raised stop is between about 0 degrees to about 15 degrees.

8. The three-dimensional implant of claim 7, where the cross-sectional area of the implant on one side of the raised stop may be the same or greater than the cross sectional area of the implant on the other side of the raised stop.

9. The three-dimensional implant of claim 1, wherein a material of the implant is bone.

10. The three-dimensional implant of claim 9, wherein the bone comprises at least one of cortical bone, and cancellous bone.

11. The three-dimensional bone implant of claim 9, wherein the bone is fully demineralized.

12. The three-dimensional bone implant of claim 9, wherein the bone is partially demineralized.

13. The three-dimensional bone implant of claim 9, wherein the bone is fully mineralized.

14. The three-dimensional bone implant of claim 9, wherein the bone is at least two of fully demineralized bone, partially demineralized bone and mineralized bone.

15. The three-dimensional bone implant of claim 1, wherein the implant is dehydrated or fully hydrated.

16. The three-dimensional bone implant of claim 1, wherein a residual moisture content of the implant is less than about 6%.

17. The three-dimensional bone implant of claim 1, wherein the at least one raised ridge on at least one surface of the body of the implant are sinusoidal-like.

18. The three-dimensional bone implant of claim 1, wherein the at least one raised ridges is substantially circular in shape.

19. The three-dimensional bone implant of claim 1, wherein the at least one raised ridges is substantially octagonal in shape.

20. A method of using the three-dimensional implant, comprising:
preparing at least one cavity in a first bone to be fused;
preparing at least one cavity in a second bone to be fused, wherein the first bone and the second bone are adjacent;
providing an implant, wherein the implant comprises:
a body;
at least one raised ridge along the body of the implant;
a raised stop along a length of the implant, wherein the raised stop comprises a first side and a second side;
a first end of the implant; and
a second end of the implant, wherein the implant comprises at least one biocompatible material;
placing the first end of the implant in the at least one cavity of the first bone until the first side of the raised stop of the implant is in contact with a surface of the first bone; and
placing the second end of the implant into the at least one cavity of the second bone until the second side of the raised stop of the implant is in contact with a surface of the second bone.

21. The method of claim 20, wherein at least one diameter of the at least one cavity of the first bone or the at least one cavity of the second bone is smaller than a cross-sectional height of the implant.

22. The method of claim 21, wherein the at least one diameter of the at least one cavity of the first bone or the at least one cavity of the second bone is between about 0.3 mm to about 0.01 mm smaller than the cross-sectional height of the implant.
